(19) United States
(12) Patent Application Publication
(10) Pub. No.: US 2011/0269802 A1
(43) Pub. Date: Nov. 3, 2011

(54) METHOD OF DELIVERY OF PERISHABLE LIQUID MIXTURES USING MIXING CAP AND CONTAINER SYSTEM

(76) Inventor: Brook LIGON, (US)

(21) Appl. No.: 12/772,664

(22) Filed: May 3, 2010

(51) Int. Cl. A61K 31/4406; A61P 3/10; A61K 31/197

(52) U.S. Cl. 514/355; 514/561

(57) ABSTRACT

Methods for preparing and delivering perishable liquid mixtures comprising at least one active ingredient. The methods use systems comprising vessels having novel barriers for segregating solvent liquids from active ingredients prior to preparation. Novel liquid mixtures having therapeutic properties are also disclosed.

### CROSS REFERENCE TO RELATED APPLICATION

This application claims priority to a provisional application, U.S. Ser. No. 61/174,199, filed Apr. 30, 2009, entitled Mixing Cap And Container System For Improved Delivery Of Perishable Liquid Mixtures, by Ligon, Brooke, which is hereby incorporated by reference.

### BACKGROUND OF THE INVENTION

1. Technical Field

The present invention relates generally to preparing and delivering perishable liquid mixtures. More particularly, the present invention relates to methods for mixing or compounding biologically active ingredients at or near the time of use by utilizing novel delivery systems.

2. Description of Prior Art

Vitamins, nutraceuticals, and other dietary supplements are frequently packaged as liquid mixtures. Likewise, common methods for delivering ingestible therapeutics often involve combining active ingredients with suitable liquid carriers. Since many active ingredients begin to degrade shortly after contact with liquid, their intended beneficial effects may significantly diminish before being ingested.

The pH, temperature, solvent type, exposure to light and air, ionic strength, and storage time of a mixture are understood to affect the stability of active ingredients. In general, the longer a labile ingredient exists in a liquid mixture, the less effective it becomes. Amino acid related dietary supplements and peptide based therapeutic agents are especially prone to degrading. If contaminated with bacteria or other microorganisms, the contaminants are likely to multiply, possibly making the mixture toxic or more rapidly diminishing the potency of active ingredients. To counter the degradation process, liquid mixtures are often refrigerated. Solutions containing degradable agents also commonly include stabilizers and preservatives. While these countermeasures are usually successful at reducing degradation they are difficult to accomplish and undesirable in many circumstances. Refrigeration is costly and may be ineffective at maintaining perishable mixtures at cold temperatures throughout their manufacturing and distribution processes. Similarly, regularly ingesting stabilizers and preservatives is prohibitive and thus products with such additives are generally less appealing to consumers when more healthful alternatives exist.

Other alternative and supplemental techniques have been developed to reduce the degradation of active ingredients in perishable liquid mixtures. When the loss of biological activity can be accurately calculated, a greater proportion of active ingredient may be provided in a product to compensate. This strategy may be satisfactory when the cost of active ingredient is minimal and the character and quantity of degradation products are readily tolerated. However, large concentrations of degradation products may noticeably precipitate or change the overall beneficial and appealing properties of the product. Some perishable liquid mixtures are lyophilized and later reconstituted immediately prior to use. Reconstitution often requires measuring a suitable amount of solvent and combining it with a lyophilized ingredient. More convenient systems provide premeasured amounts of liquid for combining with the lyophilized portion. In addition to the added processing cost, spilling or contaminating the active ingredients or diluent is an undesirable risk of lyophilized liquid delivery devices.

To improve upon these shortcomings, various devices have been invented for segregating premeasured amounts of liquid and dry or powdered ingredients until time for mixing. A popular approach involves breaking off or displacing a perforated structural barrier which functions as an ingredient chamber bottom inside the segregating device. As an example, U.S. Pat. No. 6,644,471 describes a device containing dry ingredients for inserting into the neck of a bottle. The device segregates powdered ingredients from liquid in the bottle until steps are taken to break a plunger mechanism free. As with many of these devices, the dry ingredient enters the liquid mixture accompanied by the discarded plunger.

An improvement over devices which dispose of components in the liquid mixture, U.S. Pat. No. 7,032,745 discloses a device for fitting inside the neck of a bottle in which a plastic ingredient chamber bottom is used as a penetrable barrier. The barrier is perforated along its perimeter except for a segment which functions as a hinge. When the cap is manipulated to rupture the barrier, an opening is created which allows the ingredients to fall. However, the barrier remains attached to the device along its un-perforated edge and thus only the ingredients combine with the liquid. Devices which employ easily penetrable foil or paper barriers are generally similar in function.

U.S. Pat. No. 7,464,811 avoids the problem of contaminating mixtures with discarded barrier parts by employing a cap having an ingredient chamber and multiple apertures. In a one position the apertures are covered and liquid is prevented from reaching the ingredients. In a second position, the apertures are opened so that ingredients and liquid may pass through the apertures to mix.

Although potentially feasible as perishable mixture delivery systems in limited circumstances, the previous devices have failed to become widely accepted. Aside from the hazard of ingesting discarded components, devices with small ports or openings are prone to clogging as dry ingredient and liquid are mixed. Furthermore, manufacturing multipart mechanisms capable of reliably penetrating barriers in otherwise sealed containers is relatively expensive, particularly as a result of the necessary moving parts.

A more economical and reliable approach incorporates a peelable paper, plastic, or foil barrier as is present in common beverage containers. An example of a device employing such technology to deliver liquid mixtures is U.S. patent application Ser. No. 10/819,731 which includes multiple embodiments of a simply designed and easily manufactured cap. The innermost portion of the cap holds enhancement ingredients protected by a fluid soluble seal. One embodiment includes a tabbed and peelable fluid insoluble seal. The insoluble seal is described and depicted as being disposed on the outer surface of the cap, over its opening, for purposes of sealing off the cap and preventing tampering. To prepare a liquid mixture, the fluid insoluble seal is peeled away and the cap is attached to a suitable container at least partially filled with fluid. Then the container is shaken until the soluble seal, enhancement, and fluid are mixed. While this device is contemplated for mixing and delivering ingredients such as flavorings or vitamins, it may be unsuitable for pharmaceutical applications since the quality and quantity of solvent fluid cannot be completely controlled.

While perishable liquid mixtures may be prepared and delivered advantageously by the preceding inventions, each is significantly limited. Therefore, there is a need for a simple, economical, and easy to use perishable liquid mixture delivery system capable of preventing spills and contamination while maximizing the properties of its active ingredients.

### SUMMARY OF THE INVENTION

Accordingly, an aspect of the present invention is directed to a system for improving the preparation and delivery of perishable liquid mixtures which overcomes the disadvantages of devices in the prior art. More specifically, the structure and operative features of the invention described herein provides for the reliable preparation and delivery of perishable liquids in a vessel which is easily manufactured at minimal cost. Further still, the invention disclosed herein provides an economical means for delivering safe and accurate doses of nutraceutic and pharmaceutic agents with minimum degradation products and extended shelf life. In a further aspect of the invention, methods are disclosed for delivering safe and economical perishable liquid mixtures according the system described herein. In yet a further aspect of the invention, specific therapeutic mixtures are disclosed for delivery with the systems and methods described as well as by conventional means.

One aspect of the system includes a vessel having a container portion and a removable cap. The container portion includes a neck and a rim and is generally positioned toward the bottom of the vessel relative to the cap. The cap has a closed end and an open container portion end. The cap is structured with an ingredient chamber to hold a precise quantity of active ingredient therein. The active ingredient is confined within the ingredient chamber by various means including one or more removable barriers disposed between the active ingredient and the container portion end of the cap. At least one of the barriers is in close proximity to the active ingredient and soluble in the solvent liquid. The soluble barrier is adhered, compacted, tightly fitted, or otherwise secured to its position in the cap, covering the ingredient chamber. The cap and barriers fully enclose the active ingredient and prevent the active ingredient from being spilled or contaminated while the cap is removed or manipulated during preparation of the perishable liquid mixture.

The container portion of the vessel holds a volume of solvent liquid capable of disrupting the active ingredient along with the soluble barrier, thereby removing them from their positions in the cap. Once removed, the active ingredient and the soluble barrier are incorporated with the solvent liquid. The active ingredient, solvent liquid, and soluble barrier are present in such proportion that when combined, the resulting perishable liquid mixture is substantially uniform and may easily be delivered by the vessel for ingestion. Furthermore, the active ingredient, solvent liquid, and soluble barrier are in such proportion that the biological activity of the active ingredient is sufficient to provide a desired physiological effect.

Preferably, the system vessel also comprises one or more insoluble barriers resistant to the solvent liquid. The insoluble barrier is adhered to the rim of the container portion as a sealing means or disposed inside the removable cap such that the insoluble barrier prevents the solvent liquid from contacting the soluble barrier until the insoluble barrier is removed. In either case, the insoluble barrier preferably includes an easily grasped tab to help remove the insoluble barrier from the system vessel. When the system vessel includes an insoluble barrier adhered to the rim of the container portion, it thereby prevents the solvent liquid from exiting the container portion and protects against spills or contamination. When the system vessel includes an insoluble barrier within the cap in close proximity to the soluble barrier, the insoluble barrier is adhered to a corresponding sealing surface provided in the cap and functions with the cap to completely enclose the soluble barrier and active ingredients. In preferred embodiments, the insoluble barrier is one of only two barriers in the vessel; the other being the soluble barrier positioned in the cap. Optionally, the system vessel comprises a soluble barrier accompanied by two insoluble barriers, one adhered to the cap and another adhered to the container portion.

In alternate embodiments of the invention, active ingredient is made firm or incorporated into the material of a soluble barrier such that the active ingredient is retained within the ingredient chamber of the cap without need for a separate soluble barrier. Unless manufactured with a container portion having solvent liquid these embodiments are may be provided without an insoluble barrier.

In yet other alternate embodiments of the invention the vessel does not incorporate a container portion, but rather the cap is suitably adapted to be attached to a standard container. In such embodiments the cap may comprise threads formed into its open end such that said threads may be engaged with standard threads formed onto the neck of a container.

The system is generally operated by first readying the vessel for preparing the mixture. The vessel is readied by removing the cap and peeling away all insoluble barriers to expose the soluble barrier and open the container portion. The container portion is filled with solvent liquid if such liquid is not already present. Once the solvent liquid is present and the soluble barrier is exposed, the cap is reattached to the container portion and the vessel is inverted or otherwise agitated until the solvent liquid ruptures the soluble barrier. Thereafter, the vessel is further agitated to substantially dissolve or mix the active ingredient with the solvent liquid. When substantially mixed together the active ingredient and liquid produces a perishable liquid mixture to be ingested by humans or animals in need.

It is to be understood that the foregoing and following description of the invention is intended to be illustrative and exemplary rather than restrictive of the invention as claimed. These and other aspects, advantages, and features of the invention will become apparent to those skilled in the art after review of the entire specification, accompanying figures, and claims incorporated herein.

### BRIEF DESCRIPTION OF THE DRAWINGS

FIG. 1 is a perspective view of the invention depicting the cap and upper container portion components of the system.

FIG. 2 is a perspective view of the invention depicting major components of the system and vessel.

FIG. 3 is a side perspective view of the removable cap with active ingredients and barriers in place.

FIG. 4 is a side perspective view of the invention depicting a loaded cap and corresponding upper container portion neck according to a preferred embodiment of the invention.

FIG. 5A is a side perspective view of the invention depicting the system before active ingredients have been mixed with solvent liquid.

FIG. 5B is a side perspective view of the invention depicting the system after active ingredients have been mixed with solvent liquid.

### DETAILED DESCRIPTION

Various terms relating to the compositions and methods of the present invention are used herein above and throughout the specification and claims.

As used herein, “active ingredient” 310 includes reference to substances recognized or intended to have a specific nutraceutical or pharmacological effect.

The term “co-optimizer” includes reference to substances which, when combined with active ingredient 310, improve the recognized or intended biological activity of the active ingredient 310.

The term “GABA” includes reference to gamma-aminobutyric acid in various degrees of purity including feed, food, or pharmaceutical grades.

The term “insoluble barrier” 240 as used herein includes reference to a barrier means which is substantially resistant and impenetrable to the solvent liquid 320 such that when the insoluble barrier 240 is sealably disposed between the solvent liquid 320 and the active ingredient 310, the solvent liquid 320 remains isolated from the active ingredient 310 despite prolonged contact of the solvent liquid 320 with the insoluble barrier 240.

The term “less than normal insulin production” as used herein refers to a deficient level of insulin production, such that a human or animal patient or subject will have a fasting blood glucose measurement which is at least periodically outside of the acceptable normal range.

The term “perishable liquid mixtures” 300 as used herein includes reference to liquid mixtures having at least one active ingredient 310 in a solvent liquid 320 and which can be ingested by pouring the mixture 300 from the container portion 210 of the system 100. Perishable liquid mixtures 300 as used herein are intended to include solutions, suspensions, colloidal dispersions, and emulsions.

The term “perishable liquid mixture delivery system” 100 or also synonymously “system” 100 as used herein includes reference to the vessel 200, barriers 240,230, active ingredients 310, and other substances contained therein.

The term “solvent liquid” 320 refers to liquid in the container portion 210 of the vessel 200 for combination with the active ingredient 310. The solvent liquid 320 is capable of disrupting the structure of the soluble barrier 230 and mixing with the soluble barrier 230 and active ingredient 310. The solvent liquid 320 may be present in the system 100 as manufactured or added to the container portion 210 of the vessel 200 at any time prior to preparation of the perishable liquid mixture 300.

The term “soluble barrier” 230 as used herein refers to ingestible material attached within the recess 222 of the cap 220 which is generally soluble in the solvent liquid 320. The soluble barrier 230 generally encloses active ingredients 310 in the ingredient chamber 224 of the cap 220 but may in certain embodiments consist, in part, of active ingredients 310 such that no other active ingredient 310 is present in the vessel 200. In such certain embodiments it should be understood that dissolving the soluble barrier 230 will impart adequate desired function of the active ingredient 310 to the perishable liquid mixture 300.

The term “sealing means” as used herein refers to an insoluble barrier 240 or other means which functions to prevent solvent liquid 320 from exiting the container portion 210 of the vessel 200.

The term “vessel” 200 as used herein generally includes the container portion 210, removable cap 220, and any structural features contained therein.

A major aspect of the invention includes embodiments of a perishable liquid mixture delivery system 100 for preparing liquid drinks for general consumption as well as for compounding and delivering human and veterinary pharmaceuticals. The perishable mixture delivery system 100 of the present invention is disposed and structured to avoid contamination and be easily operated without complicated instructions or training In all applications, the system 100 of the present invention provides a means for assembling perishable liquid mixtures 300 immediately prior to ingestion, thereby maximizing the properties and characteristics of the active ingredient 310 for the greatest nutritional, nutraceutical, or pharmacological effect. Furthermore, the delivery system 100 of the present invention optimally maintains the physical, chemical, and microbiological stability of active ingredients 310 with minimal need for preservatives, freeze drying, refrigeration, or the like, or expensive to manufacture components. Therefore, the system 100 may be used to combine suitable solvent liquids 320 with active ingredients 310 and deliver the resulting perishable liquid mixtures 300 more safely, efficiently, and economically than previous perishable mixture delivery devices.

Reference will now be made to presently preferred embodiments of the invention, examples of which are illustrated in the accompanying drawings.

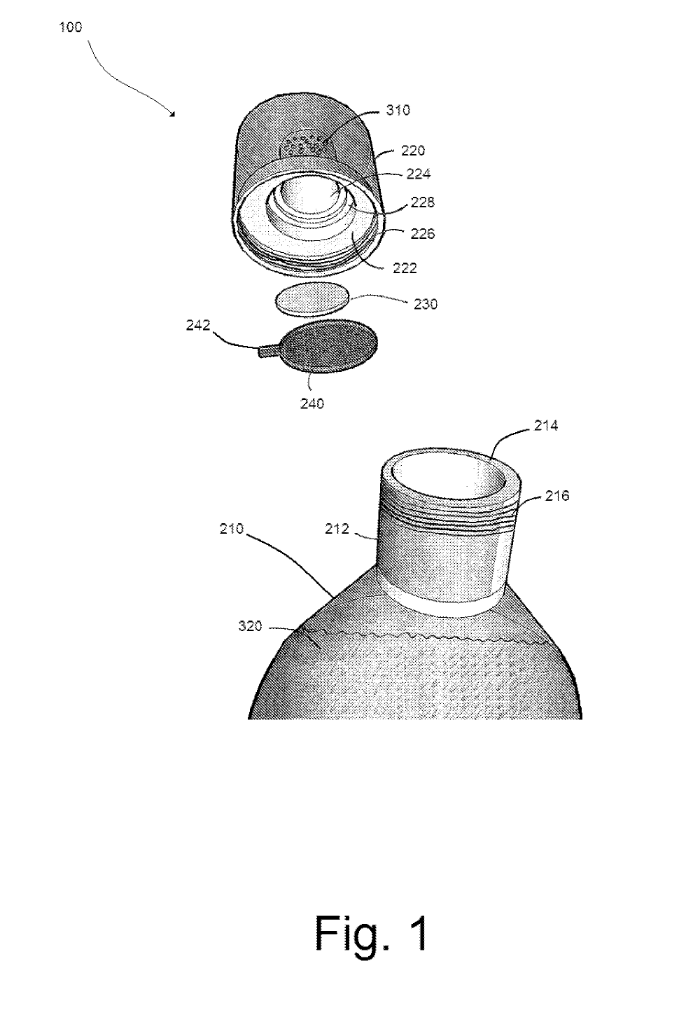

Referring to FIG. 1, a perishable liquid mixture delivery system 100 in accordance with exemplary embodiments of the present invention is described. As illustrated, the delivery system 100 comprises a vessel 200 having a substantially hollow container portion 210 adapted to be filled with solvent liquid 320 and a removable cap 220. The cap 220 and container portion 210 are coupled together by external threads 216 on the neck 212 of the container portion 210. Said threads 216 correspond in the usual manner to internal threads 226 disposed within the recess 222 of the removable cap 220. However, other suitable fastening means may be used to join the cap 220 and container portion 210 of the vessel 200. The cap 220 further includes an ingredient chamber 224 wherein active ingredient 310 is maintained separately from the solvent liquid 320 until it is desired that the perishable liquid mixture 300 be prepared or compounded. Preferably, the active ingredient 310 is in powder or granulated form. However, the active ingredient 310 may also be packed solid, held together with filler or binder or the like, or may be present substantially as a liquid or a gel. Said ingredient chamber 224 is of sufficient size and in proportion to the volume of solvent liquid 320 held the container portion 210 such that when the solvent liquid 320 and the active ingredient 310 are combined, the resulting perishable liquid mixture 300 will be biological active.

The vessel 200 of the perishable liquid mixture delivery system 100 further comprises one or more removable barriers 230,240 (FIG. 1) which functions to isolate the active ingredient 310 from the solvent liquid 320. At least one of the removable barriers 240 is effectively resistant to being disintegrated by the solvent liquid 320, and performs as an insoluble barrier 240 which remains in its position in the vessel 200 until it is peeled away or otherwise manually removed. Depending upon the properties of solvent liquid 320, the insoluble barrier 240 can be made from foil, plastic, wax paper or the like and adhered to the vessel 200 according to conventional beverage bottling methods well known to persons skilled in the art.

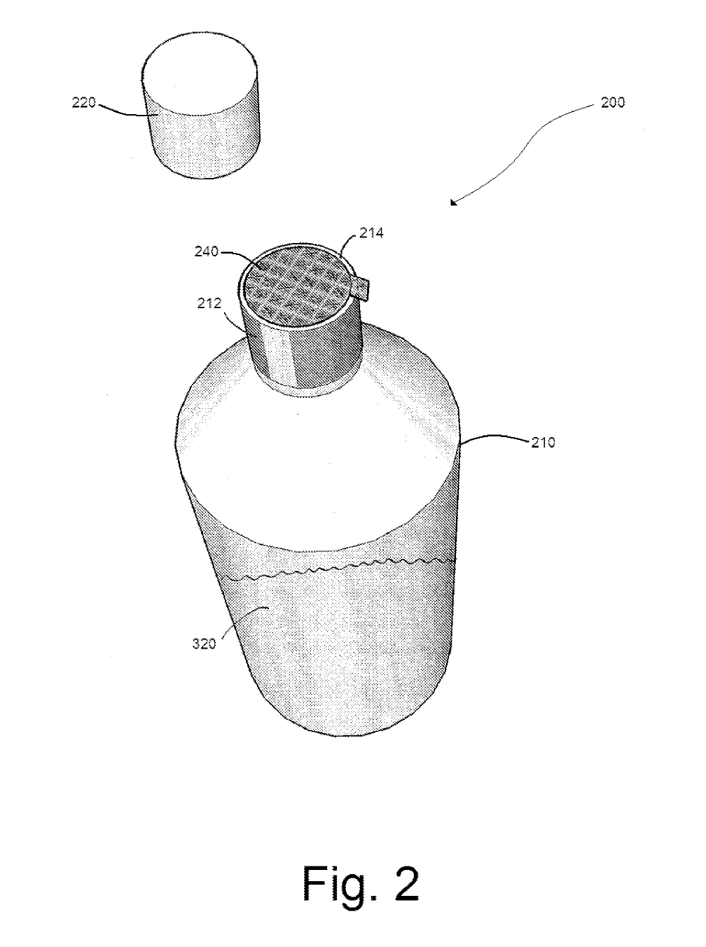

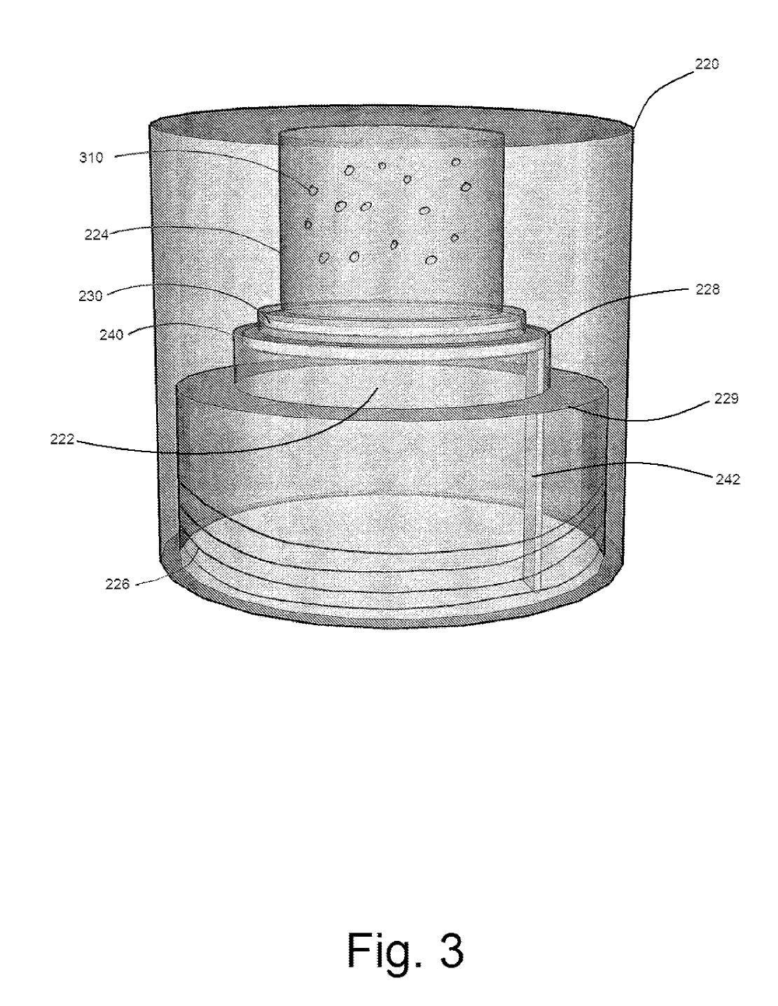

The insoluble barrier 240 must be sealably attached to a corresponding sealing surface 228 of the vessel 200 to prevent the solvent liquid 320 from contacting the active ingredient 310. In a preferred exemplary embodiment of the system 100 as depicted in FIG. 2, the insoluble barrier 240 is adhered to the rim 214 of the neck 212 of the container portion 210, thereby functioning as a sealing means and preventing the solvent liquid 320 from exiting the container portion 210. Optionally, and as in a preferred exemplary embodiment of the system 100 as depicted in FIG. 3, the insoluble barrier 240 may be disposed at other locations in the vessel 200 including but not limited to a designated sealing surface 228 within the recess 222 of the cap 220 in such a manner that the insoluble barrier 240 is disposed over and seals the ingredient chamber 224 from the solvent liquid 320. As is depicted in FIG. 3, if the insoluble barrier 240 is adhered to the cap 220 rather than the container portion 210 and the container portion 210 is without a sealing means, it is critical that the vessel 200 include a contact shoulder 229 or similarly functioning feature to sealably contact the rim 214 of the container portion 210 and prevent liquid from leaking outside the vessel 200 when the system 100 is shaken or inverted.

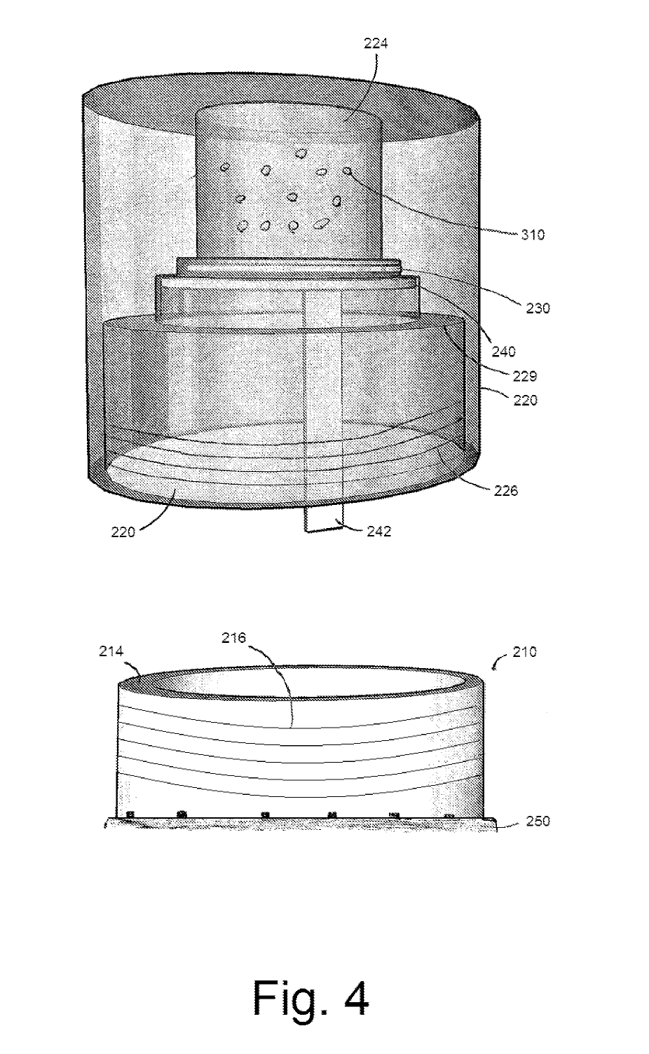

In preferred exemplary embodiments, the insoluble barrier 240 includes a tab 242 which is easily grasped so that the insoluble barrier 240 may be peeled away from its position in the vessel 200, prior to the preparation of the perishable liquid mixture 300. Also in preferred embodiments, the vessel 200 includes a soluble barrier 230 in close proximity to the active ingredient 310. The soluble barrier 230 is easily removed by the solvent liquid 320 and combines with the perishable liquid mixture 300 when dissolved. Referring now to FIG. 4, the soluble barrier 230 is retained within or adhered to the cap 220 such that it contains the active ingredient 310 inside the ingredient chamber 224 until the soluble barrier 230 is disintegrated by the solvent liquid 320. Furthermore, the soluble barrier 230 allows the cap 220 to be inverted or otherwise manipulated while separated from the container portion 210 without the risk of spilling or contaminating the active ingredient 310.

The soluble barrier 230 may be made from combinations of nontoxic and ingestible solid or semi-solid material able to be disintegrated by the solvent liquid 320. Some examples of such materials include cellulose, agar, gelatin, polyvinyl alcohol, calcium phosphate, calcium carbonate, alginate, sorbitol, mannitol, lactose, glucose, sucrose, or starch. Preferably, the soluble barrier 230 material chosen is rapidly disintegrated in aqueous solution and therefore functional with most common liquid beverages. Numerous instructive texts cover the application of such materials. Incorporated herein by reference as an example is Peter A. Williams, Handbook of Industrial Water Soluble Polymers, (1^{st} ed., Blackwell 2007).

In the exemplary embodiment as depicted in FIG. 4, sufficient active ingredient 310 to affect a desired result is held within the ingredient chamber 224 proximate to the central axis portion of the removable cap 220. The cap 220 includes internal threads 226 to mate with the external threads 216 of container portion 210 of the system 100. The active ingredient 310 is granulated and completely covered by a soluble barrier 230 adhered to the open end of the ingredient chamber 224. The cap 220 also includes an insoluble barrier 240 disposed over the soluble barrier 230 and sealably adhered to a circular sealing surface 228 within the cap 220. The container portion 210 includes a volume of solvent liquid 320 in correct functional proportion to the active ingredient 310. Also included in the recess 222 of the cap 220 and outside the ingredient chamber 224 is a contact shoulder 229 around the internal perimeter of the cap 220 for sealing with the rim 214 of the container portion 210. Thus, when the cap 220 is tightened to the container portion 210, the seal between the contact shoulder 229 and rim 214 prevents solvent liquid 320 from leaving the vessel 200. Optionally, the insoluble barrier 240 is larger in diameter than the sealing surface 228 and continues outward radially from the central axis of the cap 220 to be disposed between the contact shoulder 229 and the rim 214 such that when the cap 220 is combined with the container portion 210, the insoluble barrier 240 functions also as a sealing means to confine the solvent liquid 320 to the container portion 210 of the vessel 200. As depicted in FIG. 4, the insoluble barrier 240 is substantially round and includes a thin plastic tab 242 for pulling the insoluble barrier 240 free of the cap 220. Preferably the tab 242 is disposed outward from the central axis of the cap 220 and wound between the threads 216,226 of the vessel 200 during manufacture to avoid being made wet by the solvent liquid 320. When threaded together, the cap 220 and container portion 210 are also connected with a tamper evident band 250.

In accordance with the present invention, active ingredient 310 may optionally be in liquid form. Depending upon the nature of the active ingredient 310, adaptation of the system 100 for such purposes may require added depth or other fortification to the soluble barrier 230. Preferably, the liquid of the active ingredient 310 is immiscible with and relatively ineffective at disintegrating the soluble barrier 230 such that the soluble barrier 230 remains intact until it is disrupted by the solvent liquid 320 held in the container portion 210 of the system 100.

In a further optional embodiment, the active ingredient 310 is compacted, gelled, combined with soluble barrier 230 materials or binder, or otherwise made firm so that the active ingredient 310 will function significantly as the soluble barrier 230. More specifically, in such embodiments the active ingredient 310 is retained in or adhered to the ingredient chamber 224 and remains disposed therein until removed by contact with the solvent liquid 320. In such optional embodiments, the active ingredient 310 laden soluble barrier 230 may be covered by an insoluble barrier 240 which is manually removed. Said insoluble barrier 240 may be accompanied by a second insoluble barrier 240 disposed on the container portion 210 of the vessel 200 or may be the only insoluble barrier 240 of the system 100. In all such embodiments, the active ingredient 310 remains ingestible and substantially nontoxic despite being made firm to function as the soluble barrier 230.

In still another preferred embodiment of the present invention, the system 100 lacks a container portion 210 but includes an active ingredient 310 laden cap 220 compatible with a popular liquid drink product such as bottled water, electrolyte beverage, soda, fruit juice, or the like. In this and similar embodiments, the liquid drink product and cap 220 are fitted to one another to function together as a perishable liquid mixture delivery system 100. Referring now to the exemplary embodiment in FIG. 3, the cap 220 includes internal threads 226 corresponding to external threads of a common liquid beverage container. Said cap 220 also includes an active ingredient 310 covered by a soluble barrier 230 which may be disintegrated by the solvent liquid 320 in said common liquid beverage container in accordance with the present invention. Said cap 220 further includes an insoluble barrier 240 between the internal threads 226 of the cap 220 and the ingredient chamber 224 which is easily removed to expose the soluble barrier 230. Optionally, said soluble barrier 230 may be made especially robust and protected from mishaps during handling, shipping, and storage by appropriate packaging. In such optional embodiments said soluble barrier 230, once fitted to said common liquid beverage container, may be protected from solvent liquid 320 by an insoluble barrier 240 inherent to and typical of such drink products.

Referring generally now to all embodiments, components of the vessel 200 may be constructed of glass, metal, plastic, paper, and other materials using common existing technology. Similarly, numerous embodiments of the system 100 including its related barriers 240,230 may be manufactured using packaging processes, materials, and methods well known to persons in the consumer beverage, nutritional supplement, and pharmaceutical packaging fields. Examples of readily available guides containing such information include: Philip D. Rufe, Fundamentals of Manufacturing, (2d ed., SME 2001); Geoff A. Giles, Handbook of Beverage Packaging, (1^{st} ed., Wiley-Blackwell 1999); Philip R. Ashurst, Chemistry and Technology of Soft Drinks and Fruit Juices, (2d ed., Blackwell 2005); Dorothy A. G. Senior, Nick Dege, Technology of Bottled Water, (2d ed., Blackwell 2005); and George F. Chrader, Ahmad K Elshennawy, Lawrence E. Doyle, Manufacturing Process and Materials, (4^{th} ed., SME 2000) which are herein incorporated by reference.

In a further aspect of the invention, methods are provided for delivering perishable liquid mixtures 300 in accordance with the perishable liquid mixture delivery system 100 described herein. The structural and operative features of the system 100 of the present invention allow for preparation and delivery which is unique and distinguishable from known or conventional mixture delivery steps. Moreover, by utilizing a self contained system 100 or, optionally, a protected soluble barrier 230, the operator is afforded an easy, reliable, and relatively foolproof method for delivering perishable liquid mixtures 300 under the most challenging environments.

Perishable liquid mixtures 300 prepared, compounded, or otherwise combined by the system 100 may include solutions, suspensions, emulsions, or colloidal dispersions. If delivered as a nutraceutical or pharmaceutical, the perishable liquid mixture 300 produces desired physiologic effects.

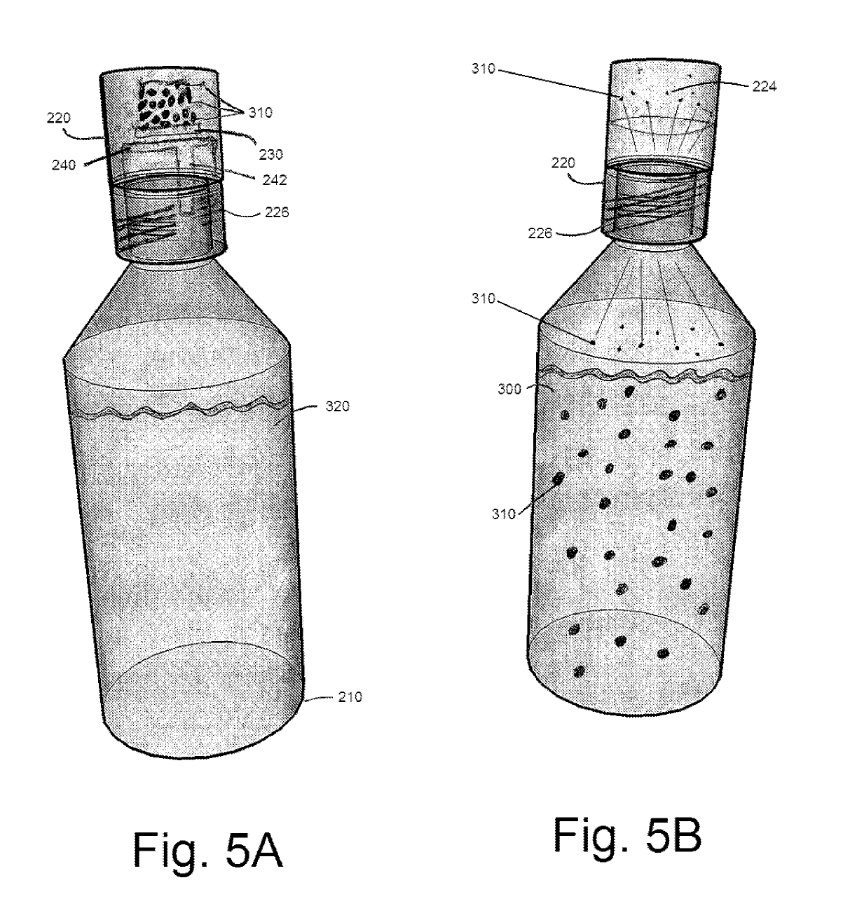

Referring now to FIGS. 5A and 5B, a perishable liquid mixture 300 may be prepared by positioning the vessel 200 upright and turning the cap 220 sufficiently with respect to the container portion 210 so that the cap 220 and container portion 210 are substantially disconnected. With the insoluble barrier 240 evident in the recess 222 of the cap 220, the tab 242 is pulled until the insoluble barrier 240 is completely removed, thereby exposing the soluble barrier 230. Next, the cap 220 is reattached to the container portion 210 and the vessel 200 is shaken until the soluble barrier 230 is disrupted and active ingredients 310 are mixed with the solvent liquid 320. Once the active ingredient 310 is adequately integrated with the solvent liquid 320, the cap 220 is again removed to deliver the resulting perishable liquid mixture 300. In a preferred embodiment, the cap 220 includes a valve means whereby the cap 220 may remain in place as the perishable liquid mixture 300 is ingested. In any case, the perishable liquid mixture 300 is preferably ingested shortly after preparation to maximize its beneficial effects.

Referring again to the exemplary embodiment of FIG. 3, wherein the cap 220 is intended to be fitted with a liquid drink product to function together as the system 100, the perishable liquid mixture 300 is prepared by first removing the insoluble barrier 240 disposed between the soluble barrier 230 and the internal threads 226 well inside the recess 222 of the system 100 cap 220. Next, with the soluble barrier 230 exposed, the cap 220 is joined to an opened drink product having adequate solvent liquid 320 volume and external threads compatible with the cap 220 threads 226. The vessel 200 is then shaken or inverted to disintegrate the soluble barrier 230 and combine the active ingredient 310 with solvent liquid 320. Agitation of the vessel 200 should continue until the active ingredient 310 and soluble barrier 230 are substantially evenly distributed throughout the solvent liquid 320. The resulting liquid mixture 300 is delivered immediately by simply removing the cap 220 and drinking the entire contents of the container portion 210. Optionally, the contents may be ingested periodically within a time frame akin to the expected stability of the mixture 300.

In yet a further aspect of the invention, perishable liquid mixtures 300 are provided for delivery by the system 100. Such perishable liquid mixtures 300 comprise active ingredients 310 labile in solution or liquid carriers and include nutritional supplements such as creatine, GABA, and melatonin; as well as recognized therapeutic pharmacological agents such as antibiotics, analgesics, antiemetics, peptide hormones, anti-inflammatories (such as glucosteroids or non-steroidal anti-inflammatories, neurotransmitters like dopamine and dopamine agonists).

More specifically, a perishable liquid mixture 300 is provided having beneficial therapeutic effect for persons and animals deficient in homeostatic insulin production.

By way of example, a preferred embodiment of the present invention includes the preparation and delivery of a perishable liquid mixture 300 comprising gamma-aminobutyric acid (GABA) as its active ingredient 310. The GABA may be isolated and purified as feed, food, nutraceutical or pharmaceutical grade for combination with a suitable solvent liquid 320, according to the nature of the patient or subject ingesting the mixture 300. The solvent liquid 320 may be any ingestible liquid suitable for mixing with GABA, including but not limited to potable water. The solvent liquid 320 may be water alone, or may also include flavorings, other nutrients, or co-optimizers. The GABA containing perishable liquid mixture 300 may be prepared and delivered according to the system 100 of the present invention or may be mixed and delivered according to conventional methods. In any case, the GABA should be mixed with solvent liquid 320 immediately prior to or near the time of consumption in order to maximize its beneficial effect. The GABA mixture 300 may be ingested all at once or in small portions throughout an extended period of time.

To be therapeutically effective, GABA is administered to patients or subjects in doses of between 0.6 and 60 milligrams per kilogram of body weight. GABA is significantly soluble in water, therefore practical proportions of GABA active ingredients 310 to aqueous solvent liquids 320 are between 5 and 50 milligrams per milliliter, depending on the type and amounts of other solutes also present.

The procedures set forth below are used for administering GABA for therapeutic effect in diabetic canine subjects having less than normal insulin production.

### EXAMPLE 1

### Preparation and delivery of GABA to Canine Subjects Deficient in Insulin Production using the system 100 of the invention herein.

The procedure comprises three basic steps: 1) GABA is provided in an ingredient chamber 224 of a perishable liquid delivery system 100, separate from a proportional volume of potable water also contained within the system 100; 2) a GABA perishable liquid mixture 300 is prepared by i) first removing the cap 220 and peeling away the insoluble barriers 240, and ii) the GABA is then dissolved by reattaching the cap 220 and inverting the container portion 210 according to the invention described herein; and 3) the resultant mixture 300 is then delivered to the subject after once again removing the cap 220.

A. Loading of the perishable mixture delivery container portion 210.

A vessel 200 is provided having a threaded 216 container portion 210 and corresponding cap 220. The cap 220 includes threads 226, an ingredient chamber 224, a sealing surface 228, and a contact shoulder 229.

Approximately 750 grams of GABA processed to approximately 99% purity is mixed with 500 grams of acacia gum binder and compacted into the active ingredient chamber 224 of the inverted cap 220 to act as a soluble barrier 230, according to the invention described in the specification herein. The chamber 224 is then covered by an insoluble barrier 240 of paper reinforced foil adhered to a sealing surface 228 of the cap 220 with warmed gelatin and allowed to cool. The container portion 210 of the delivery system 100 is filled with approximately 100-300 mls. of filtered potable water substantially free of microorganisms and pyrogens. Under essentially sterile conditions, an additional foil and paper barrier 240 prepared with warm gelatin is applied over the opening of the container portion 210 and allowed to cool. The cap 220 is joined to the container portion 210 and tightened.

B. Preparing the perishable liquid mixture 300.

At or near the time that the subject is to ingest a dose of GABA, the cap 220 is removed from the container portion 210 and both insoluble barriers 240 are removed. The cap 220 is then reattached to the container portion 210 and shaken gently until the soluble barrier 230 containing GABA is substantially dissolved.

C. Delivering the perishable liquid mixture 300.

A canine subject in need of treatment for diabetes is confined in an environment having no access to water other than from a designated dish. The mixture 300 is delivered by removing the cap 220 from the container portion 210 of the system 100 and pouring the mixture 300 into the dish. The subject is prevented from spilling the mixture 300 and observed until substantially all of the mixture 300 is ingested.

### EXAMPLE 2

### Preparation and Delivery of GABA to Canine Subjects Deficient in Insulin Production using conventional means.

METHODS: GABA (99% pure from Sigma-Aldrich) at a dose of 30 mg/kg was brought into solution in 200 milliliters of water daily so that the diabetic, insulin dependent canine test subject consumed the dose within approximately four hours, based on average consumption of water observed by owners. Fasting blood glucose and fructosamine were monitored once a month. Each canine subject served as its own control, with monitoring of blood glucose and fructosamine off treatment recorded the first two months followed by monitoring while on GABA therapy.

RESULTS: Insulin dependent test subjects began to demonstrate improved glycemic control within two weeks of consuming GABA diluted in water, observations continued over four months with continued improvement in glycemic control over that period. The fasting blood glucose in the first test subject declined by 49.6% after two months of treatment. Fasting blood glucose in the second test subject also declined by 50% after two months of treatment. Similarly, both subjects demonstrated significantly lower concentrations of blood fructosamine as a result of treatment with GABA.

In preferred embodiments of perishable liquid mixtures 300 in accordance with the invention, active ingredients 310 are accompanied by co-optimizers to improve the biological activity of the active ingredient 310. Co-optimizers may include any substance which improves the effectiveness of the active ingredient 310 and comprise proteins, peptides, amino acids, vitamins, and recognized organic and inorganic cofactors. The co-optimizer is dissolved in a solvent fluid 320 and separated from active ingredients 310 in a perishable liquid mixture system 100. Optionally, the co-optimizer is in dry form and mixed directly with the active ingredient 310.

In a particular preferred embodiment, GABA is combined with water as a solvent liquid 320. The water incorporates niacinamide (or nicotineamide, or nicotinic acid amide) as a co-optimizer synergistic with GABA. Together in the perishable liquid mixture 300 the GABA and niacinamide (or nicotineamide, or nicotinic acid amide) are therapeutic for an absence or deficit of insulin in diabetic patients or subjects. The GABA and co-optimizer may be delivered by a perishable liquid mixture delivery system 100 according to the invention herein or may be delivered by more conventional means.

As with all the embodiments of the invention, systems 100 incorporating active ingredients 310, solvent liquids 320, and co-optimizers are manufactured and packaged using good manufacturing practices. In particular, active ingredients 310, solvent liquids 320, and co-optimizers are manufactured as substantially sterile and pathogen and pyrogen free. Depending on the combination of co-optimizer and solvent liquid 320, preservatives such as citric acid, sodium citrate, methylparaben or antioxidants and amino acids may be used. Similarly, common stabilizers such as dextran, albumin, glycerol, inositol, polyvinyl alcohol, mannitol, and maltose, may be necessary to maintain the physical, chemical, and biological properties of co-optimizers present in the solvent liquid 320.

What has been described and illustrated herein is a preferred embodiment of the invention along with some it its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

What is claimed is:

1. A method of treating a mammal with less than normal insulin production by using an active ingredient delivery system for providing a perishable liquid mixture comprising at least one active ingredient suitable for treating a mammal with less than normal insulin production, said active ingredient delivery system comprising a cap suitably adapted to be removably fitted to a container, said cap containing said at least one active ingredient and having a soluble barrier to retain said active ingredient within said cap, said container suitably adapted to contain a solvent liquid, said method comprising the steps of:
A. preparing components of the perishable liquid mixture prior to the time of treatment by
i. placing a known amount of said at least one active ingredient into said cap, and
ii. placing a known amount of the solvent liquid into the container;
B. fitting said cap onto said container;
C. preparing a known dose of the perishable liquid mixture immediately prior or close to the time the perishable liquid mixture is to be ingested by bringing said solvent liquid into contact with said soluble barrier in order to breach said soluble barrier causing the combining of said active ingredient with said solvent liquid within the container;
D. delivering the perishable liquid mixture to said mammal until substantially all of the perishable liquid mixture is ingested; and
E. monitoring said mammal over the course of therapy to detect improvement in glycemic control.

2. The method of claim 1 wherein said at least one active ingredient comprises GABA.

3. The method of claim 2 wherein said therapeutic mixture further comprises a co-optimizer.

4. The method of claim 3 wherein said co-optimizer is niacinamide.

5. A method of treating a mammal with less than normal insulin production by using a perishable liquid delivery system for delivering a perishable liquid mixture having at least one active ingredient suitable for treating a mammal with less than normal insulin production, said perishable liquid delivery system suitable for preparing and dispensing perishable liquids and comprising a vessel having a container portion fitted with a removable cap, said cap containing said at least one active ingredient and having a soluble barrier, said method comprising the step of:
A. delivering said mixture to the mammal.

6. The method of claim 5 wherein said at least one active ingredient comprises GABA.

7. The method of claim 6 wherein said therapeutic mixture further comprises a co-optimizer.

8. The method of claim 7 wherein said co-optimizer is niacinamide.

9. The method of claim 5 comprising the further steps of:
A′. preparing components of the perishable liquid mixture prior to the time of treatment by placing a known amount of the at least one active ingredient and a known amount of a solvent liquid into the perishable liquid delivery system;
A″. preparing a known dose of the perishable liquid mixture immediately prior or close to the time the perishable liquid mixture is to be ingested by bringing said solvent liquid into contact with said soluble barrier in order to disrupt said soluble barrier causing the combining of said active ingredient with said solvent liquid within the container portion of the perishable liquid delivery system; and
B. monitoring said mammal over the course of therapy to detect improvement in glycemic control;
wherein delivering the perishable liquid mixture to said mammal in step A is accomplished by using the perishable liquid delivery system until substantially all of the perishable liquid mixture is ingested by the mammal; and
steps A′ and A″ are performed before step A and step B is performed after step A.

10. The method of claim 9 wherein said at least one active ingredient comprises GABA.

11. The method of claim 10 wherein said therapeutic mixture further comprises a co-optimizer.

12. The method of claim 11 wherein said co-optimizer is niacinamide.

13. The method of claim 5 wherein said container portion contains a volume of solvent liquid in specific proportion to the amount of said active ingredient, said method comprising the further step of:
A′. combining said active ingredient with said solvent liquid into a mixture, wherein step A′ is performed before step A.

14. The method of claim 13 wherein said at least one active ingredient comprises GABA.

15. The method of claim 14 wherein said therapeutic mixture further comprises a co-optimizer.

16. The method of claim 15 wherein said co-optimizer is niacinamide.

17. A method of using a perishable liquid delivery system for treating a mammal with less than normal insulin production, said perishable liquid delivery system suitable for preparing and dispensing perishable liquids and comprising a vessel having a container portion fitted with a removable cap, said cap containing at least one active ingredient and having a soluble barrier, said method comprising delivering to the mammal by use of the perishable liquid delivery system a therapeutic mixture comprising said at least one active ingredient, wherein said at least one active ingredient comprises GABA.

18. The method of claim 17 wherein said therapeutic mixture further comprises a co-optimizer.

19. The method of claim 18 wherein said co-optimizer is niacinamide.
